(19) United States
(12) Patent Application Publication
(10) Pub. No.: US 2005/0281875 A1
(43) Pub. Date: Dec. 22, 2005

(54) Promethazine containing dosage form

(75) Inventors: Viswanathan Srinivasan, The Woodlands, TX (US); Ralph Brown, Southlake, TX (US); David Brown, Colleyville, TX (US); Himanshu Patel, North Richland Hills, TX (US)

Correspondence Address: GREENBLUM & BERNSTEIN, P.L.C., 1950 ROLAND CLARKE PLACE, RESTON, VA, 20191, US

(73) Assignee: Sovereign Pharmaceuticals, Ltd., Fort Worth, TX (US)

(21) Appl. No.: 11/102,725

(22) Filed: Apr. 11, 2005

Related U.S. Application Data
(63) Continuation-in-part of application No. 10/736,902, filed on Dec. 17, 2003.

(51) Int. Cl. A61K 31/5415; A61K 9/24

(52) U.S. Cl. 424/472; 514/225.8

(57) ABSTRACT

A pharmaceutical dosage form which comprises promethazine and/or a pharmaceutically acceptable salt thereof. The dosage form is capable of providing a promethazine plasma concentration within a therapeutic range for at least about 24 hours per single dose. This Abstract is neither intended to define the invention disclosed in this specification nor intended to limit the scope of the invention in any way.

### CROSS-REFERENCE TO RELATED APPLICATIONS

The present application is a continuation-in-part of U.S. patent application Ser. No. 10/736,902, filed Dec. 17, 2003, the entire disclosure of which is expressly incorporated by reference herein.

### BACKGROUND OF THE INVENTION

1. Field of the Invention

The present invention relates to a pharmaceutical dosage form which contains promethazine and/or a pharmaceutically acceptable salt thereof, optionally in combination with at least one additional active ingredient or drug. The dosage form is capable of providing a promethazine plasma concentration within a therapeutic range for at least about 24 hours per single dose when administered to a subject in need thereof. The present invention also relates to methods of alleviating conditions which can be alleviated by promethazine and the optional additional active ingredient.

2. Discussion of Background Information

Promethazine is a phenothiazine derivative which possesses antihistaminic, sedative, antimotion-sickness, antiemetic, and anticholineric effects. It is used, for example, for the amelioration of allergic reactions, the treatment of motion sickness and the prevention and control of nausea and vomiting associated with certain types of anesthesia and surgery. However, a single dose of promethazine provides relief of the indicated symptoms for only about 12 hours, requiring the patient to take another dosage form in order to maintain the effects of promethazine for the balance of the 24 hour day.

Further, allergic reactions, in particular, which can be treated or ameliorated with promethazine are often accompanied by conditions which can not satisfactorily be ameliorated or treated with promethazine, but may be treated or ameliorated by other drugs, e.g., expectorants, mucus thinning drugs, decongestants, antitussives, and/or analgesics. However, a single dose of promethazine can provide a therapeutically effective plasma concentration for an extended period of time, up to 12 hours, whereas a single dose of other drugs will often provide a therapeutically effective plasma concentration for a considerably shorter period. For example, a single dose of an expectorant such as guaifenesin will usually provide relief for only about one hour, and decongestants, antitussives, and analgesics usually provide relief for about 4 to 8 hours per single dose. As a result, there appears to be virtually no benefit in combining promethazine and any such drug with a noticeably shorter effective period in a single dosage form. With a corresponding combination, the promethazine would still provide the desired therapeutic effect when the other drug has long ceased to be effective and would have to be administered again.

It would be desirable if patients suffering from, e.g., respiratory congestion, inflammation of the respiratory mucosa and sinus cavities, weeping eyes, rhinorrhea, Eustachian Tube congestion, cough, nausea, aching joints, nausea and related gastrointestinal complaints, headache and fever and related symptoms, for which promethazine alone or in combination with another drug, for example, pseudoephedrine, is indicated, could obtain relief by ingesting only one dosage per 24 hour period. Not only would this convenient dosage form increase compliance with ambulatory patients, it would also simplify dosage for patients in institutional care, such as nursing homes and hospitals, where nurses are required to administer such dosages. For example, a once a day dosage in such institutions would reduce nursing staff responsibilities and time by 50% for the purpose of administering these agents.

It would also be desirable if patients suffering from, e.g., the above-mentioned conditions for which promethazine is indicated, would also obtain relief, over a similar time period, from one or more conditions for which drugs different from promethazine are indicated, by administering a single dose of a dosage form such as, e.g., a tablet, liquid, syrup, suspension, capsule and the like which provides both promethazine and one or more other drugs.

### SUMMARY OF THE INVENTION

The present invention provides a pharmaceutical dosage form comprising promethazine and/or a pharmaceutically acceptable salt thereof, which dosage form is capable of providing a promethazine plasma concentration within the therapeutic range for at least about 24 hours per single dose.

In one aspect, the dosage form of the present invention may be capable of providing relief from allergy symptoms in a patient in need thereof for at least about 24 hours per single dose.

In another aspect, the dosage form may comprise at least two promethazine formulations which exhibit different release profiles, for example, an immediate release formulation and a controlled release formulation.

In yet another aspect, the dosage form may comprise a solid dosage form such as, e.g., a tablet, a capsule or a caplet. For example, the dosage form may comprise a bi-layered tablet. By way of non-limiting example, this bi-layered tablet may comprise an immediate release layer and a controlled release layer. In one aspect, each of the two layers, e.g., an immediate release layer and a controlled release layer, may comprise at least about 25 mg (e.g., at least about 40 mg, or at least about 50 mg) of promethazine hydrochloride or an equivalent amount of at least one other pharmaceutically acceptable salt of promethazine (which term is intended to include promethazine free base) and/or the bi-layered tablet may comprise a total of at least about 70 mg (e.g., at least about 80 mg, or at least about 90 mg) of promethazine hydrochloride or an equivalent amount of at least one other pharmaceutically acceptable salt of promethazine and/or the controlled release layer and/or the immediate release layer may comprise at least about 40 mg of promethazine hydrochloride or an equivalent amount of at least one other pharmaceutically acceptable salt of promethazine.

In a still further aspect, the dosage form of the present invention may comprise at least about 70 mg, e.g., at least about 90 mg, of promethazine hydrochloride or an equivalent amount of at least one other pharmaceutically acceptable salt of promethazine per single dose.

In another aspect, the dosage form of the present invention may be capable of providing a promethazine plasma concentration within the therapeutic range within not more than about 1 hour (e.g., within not more than about 30 minutes) following ingestion thereof.

In yet another aspect, the dosage form may comprise at least one further drug. By way of non-limiting example, the at least one further drug may be selected from one or more of decongestants, antitussives, expectorants, mucus thinning drugs and analgesics, e.g., from one or more of phenylephrine, pseudoephedrine, phenylephrine, pseudoephedrine, codeine, dihydrocodeine, hydrocodone, dextromethorphan, carbetapentane, guaifenesin, acetaminophen, aspirin, ibuprofen, naproxen, oxycodone, morphine and hydromorphone, including pharmaceutically acceptable salts thereof. Also, the dosage form may be capable of providing a plasma concentration of the at least one further drug within a therapeutic range for at least about the same time for which it is capable of providing a plasma concentration of promethazine, e.g., for at least about 24 hours per single dose. In a further aspect, this dosage form may be capable of providing a plasma concentration within a therapeutic range of promethazine over a period which is coextensive with at least about 70%, e.g., at least about 90%, of the period over which the dosage form is capable of providing a plasma concentration within a therapeutic range of the at least one further drug. In a still further aspect, the plasma half-life of the at least one further drug may be shorter than the plasma half-life of promethazine by at least about 3 hours (e.g., by at least about 4 hours, at least about 5 hours, or at least about 6 hours).

The present invention also provides a pharmaceutical dosage form which comprises (a) promethazine and/or a pharmaceutically acceptable salt thereof in a first form or layer and (b) promethazine and/or a pharmaceutically acceptable salt thereof in a second form or layer which is different from the first form or layer. The dosage form releases the promethazine (b) over a different period and/or at a different rate than the promethazine (a) and the dosage form is capable of providing a promethazine plasma concentration within the therapeutic range for at least about 24 hours per single dose.

In one aspect, the dosage form may be a multi-layered tablet which comprises at least one immediate release layer and at least one controlled release layer wherein at least one of these layers comprises promethazine and/or a pharmaceutically acceptable salt thereof and wherein at least one of the layers comprises at least one further drug.

The present invention also provides a pharmaceutical dosage form which comprises promethazine and/or a pharmaceutically acceptable salt thereof and pseudoephedrine and/or a pharmaceutically acceptable salt thereof, wherein the dosage form is capable of providing plasma concentrations within the therapeutic ranges of both promethazine and pseudoephedrine for at least about 24 hours per single dose.

In one aspect, the dosage form may comprise a solid dosage form, for example, a multi-layered tablet.

In another aspect, the dosage form may be capable of providing promethazine and pseudoephedrine plasma concentrations within the respective therapeutic ranges within not more than about 1 hour (e.g., within not more than about half an hour) following ingestion thereof.

In yet another aspect, the dosage form may comprise promethazine hydrochloride and pseudoephedrine hydrochloride.

In a still further aspect, the dosage form may comprise at least about 70 mg (e.g., at least about 80 mg, at least about 90 mg, or at least about 100 mg) of promethazine hydrochloride or an equivalent amount of at least one other pharmaceutically acceptable salt of promethazine and/or at least about 180 mg (e.g., at least about 200 mg, at least about 220 mg, or at least about 240 mg) of pseudoephedrine hydrochloride or an equivalent amount of at least one other pharmaceutically acceptable salt of pseudoephedrine per single dose.

In a still further aspect, the dosage form may comprise at least one further drug (i.e., in addition to promethazine and pseudoephedrine).

In yet another aspect, a dosage form according to the present invention, including the various aspects thereof, may be associated with instructions to administer the dosage form once every 24 hours.

The present invention also provides a method of alleviating one or more conditions which can be alleviated by administration of promethazine and, optionally, by administration of a drug which is at least one of a decongestant, antitussive, expectorant, mucus thinning drug and analgesic, in particular, by administration of pseudoephedrine. This method comprises the administration of a pharmaceutical dosage form of the present invention, including the various aspects thereof, to a subject in need thereof. For example, the condition that can be alleviated by administration of promethazine may comprise an allergic reaction.

In one aspect of this method, the dosage form may be administered not more than about once every 24 hours.

The present invention also provides a process for making a pharmaceutical dosage form of the present invention. This process comprises preparing a first composition which comprises promethazine or a pharmaceutically acceptable salt thereof and a second composition which comprises pseudoephedrine or a pharmaceutically acceptable salt thereof, and combining the first and second compositions, for example, by a method which comprises the use of a tablet press.

In addition to the above, the present invention also provides a pharmaceutical dosage form which comprises a first drug selected from promethazine and pharmaceutically acceptable salts thereof, and at least one second drug. The dosage form is capable of providing a plasma concentration within the therapeutic range of the at least one second drug over a period which is coextensive with at least about 70% of the period over which the dosage form is capable of providing a plasma concentration within the therapeutic range of the first drug.

In one aspect of the dosage form, the at least one second drug is preferably selected from decongestants, antitussives, expectorants, mucus thinning drugs, analgesics and antihistamines. For example, the at least one second drug may comprise one or more antitussives such as, e.g., codeine, dihydrocodeine, hydrocodone, dextromethorphan and pharmaceutically acceptable salts thereof, and/or the at least one second drug may comprise one or more decongestants such as, e.g., phenylephrine, pseudoephedrine and pharmaceutically acceptable salts thereof, and/or the at least one second drug may comprise one or more expectorants, e.g., guaifenesin or a pharmaceutically acceptable salt thereof.

In another aspect of the dosage form, the first drug may comprise promethazine hydrochloride.

In yet another aspect, the plasma half-life of the at least one second drug may be shorter than the plasma half-life of promethazine by at least about 3 hours, e.g., by at least about 4 hours, or by at least about 6 hours.

In a still further aspect, the period of a plasma concentration within the therapeutic range of the at least one second drug may be coextensive with at least about 80%, e.g., at least about 90%, or at least about 95%, of the period of a plasma concentration within the therapeutic range of promethazine.

In another aspect, the dosage form may be a tablet. For example, the tablet may have at least two layers. Preferably, the tablet is a bi-layered tablet.

In yet another aspect, the dosage form comprises a solution or a suspension.

The present invention also provides a bi-layered tablet which comprises two layers. The first layer comprises promethazine and/or a pharmaceutically acceptable salt thereof. The second layer comprises at least one additional drug which is selected from decongestants, antitussives, expectorants, mucus thinning drugs, analgesics and antihistamines. The bi-layered tablet provides a plasma concentration within the therapeutic range of the at least one additional drug over a period which is coextensive with at least about 70% of the period over which the bi-layered tablet provides a plasma concentration within the therapeutic range of the promethazine.

In one aspect of the bi-layered tablet, the second layer may comprise one or both of phenylephrine and pseudoephedrine, including pharmaceutically acceptable salts thereof.

In another aspect, the first layer may comprise promethazine hydrochloride and the second layer may comprise two or more of phenylephrine, pseudoephedrine, chlorpeniramine and pharmaceutically acceptable salts thereof.

In yet another aspect, the first layer may comprise promethazine or a pharmaceutically acceptable salt thereof as the only active ingredient. For example, promethazine hydrochloride may be the only active ingredient in the first layer.

In a still further aspect of the bi-layered tablet of the present invention, the period of a plasma concentration within the therapeutic range of the at least one second drug may be coextensive with at least about 80%, e.g., at least about 90%, of the period of a plasma concentration within the therapeutic range of promethazine.

In a still further aspect of the bi-layered tablet, the first layer preferably is an immediate release layer and/or the second layer is a controlled release layer.

In another aspect, the first layer of the bi-layered tablet may contain from about 0.1 mg to about 100 mg, e.g., from about 5 mg to about 60 mg, preferably from about 25 mg to about 50 mg, of promethazine hydrochloride or an equivalent amount on at least one other pharmaceutically acceptable salt of promethazine.

In yet another aspect of the bi-layered tablet, the second layer thereof may be a controlled release layer and may contain (i) from about 1 mg to about 90 mg of phenylephrine hydrochloride or an equivalent amount of any other pharmaceutically acceptable salt of phenylephrine; and/or (ii) from about 1 mg to about 240 mg of pseudoephedrine hydrochloride or an equivalent amount of any other pharmaceutically acceptable salt of pseudoephedrine.

The present invention also provides a multi-layered tablet which comprises at least a first layer and a second layer. The first layer comprises promethazine and/or a pharmaceutically acceptable salt thereof and the second layer is a controlled release layer and comprises at least one drug which is selected from decongestants, antitussives, expectorants, mucus thinning drugs, analgesics and antihistamines.

In one aspect, the first layer of the multi-layered tablet may be an immediate release layer. In another aspect, the first layer may comprise promethazine hydrochloride.

In yet another aspect, the first layer may contain promethazine or a pharmaceutically acceptable salt thereof as the only active ingredient.

In a still further aspect of the multi-layered tablet of the present invention, the second layer preferably comprises one or more, e.g., at least two, of codeine, dihydrocodeine, hydrocodone, dextromethorphan, phenylephrine, pseudoephedrine, guaifenesin, and chlorpheniramine, including pharmaceutically acceptable salts thereof.

In another aspect of the multi-layered tablet, the at least one drug in the second layer may have a plasma half-life which is shorter by at least about 3 hours than the plasma half-life of promethazine in the first layer.

In another aspect, the first layer may comprise promethazine hydrochloride and the multi-layered tablet may provide a plasma concentration within a therapeutic range of the at least one drug in the second layer over a period which is coextensive with at least about 80% of the period over which the multi-layered tablet provides a plasma concentration within the therapeutic range of promethazine.

In a still further aspect of the multi-layered tablet, the at least one drug in the second layer may comprise one or more of phenylephrine, pseudoephedrine, chlorpheniramine and pharmaceutically acceptable salts thereof.

The present invention also provides a liquid dosage form which comprises (a) promethazine and/or a pharmaceutically acceptable salt thereof and (b) promethazine and/or a pharmaceutically acceptable salt thereof and/or at least one drug which is selected from decongestants, expectorants, mucus thinning drugs, antitussives and analgesics. If the at least one drug is present, this liquid dosage form provides a plasma concentration within the therapeutic range of the at least one drug of component (b) over a period which is coextensive with at least about 70% of the period over which the liquid dosage form provides a plasma concentration within the therapeutic range of promethazine.

In one aspect, the liquid dosage form may comprise a suspension.

In another aspect, at least a part of component (a) and/or of component (b) may be present as a complex with a complexing agent. For example, the complexing agent may comprise an ion-exchange resin such as, e.g., (sodium) polystyrene sulfonate.

In a still further aspect, the suspension may comprise particles of a complex of at least a part of component (b) with an ion-exchange resin, which particles are provided, at least in part, with a controlled release coating. This controlled release coating may comprise an organic polymer, e.g., a (meth)acrylate polymer.

The present invention also provides a method of alleviating (e.g., treating) one or more conditions which can be alleviated by administration of promethazine and a drug which is a decongestant, antitussive, expectorant, mucus thinning drug, and/or an analgesic. The method comprises administering any of the pharmaceutical dosage forms discussed above, including the various aspects thereof, to a subject in need thereof.

In one aspect of the method, the condition which can be alleviated by administration of promethazine comprises an allergic reaction.

In another aspect, the dosage form is preferably administered not more than about 3 times per day, e.g., twice per day or once per day.

The present invention also provides a process for making any of the pharmaceutical dosage forms discussed above, including the various aspects thereof. This method comprises the preparation of a first composition which comprises promethazine and/or a pharmaceutically acceptable salt thereof and the preparation of a second composition which comprises promethazine and/or a pharmaceutically acceptable salt thereof and/or at least one second drug, and the combining of the first and the second compositions to form the dosage form.

In one aspect of the process, the first and second compositions may be combined by using a tablet press.

The present invention furthermore provides a pharmaceutical dosage form which comprises (a) a first drug which is an antihistamine and has a first plasma half-life and (b) at least one second drug which is selected from decongestants, antitussives, expectorants, mucus thinning drugs, analgesics and antihistamines and has a second plasma half-life which differs from the first plasma half-life by at least about 3 hours. This dosage form provides a plasma concentration within a therapeutic range of the at least one second drug over a period which is coextensive with at least about 70% of the period over which the dosage form provides a plasma concentration within the therapeutic range of the first drug.

In one aspect, the first half-life may be longer by at least about 4 hours than the half-life of the at least one second drug.

In another aspect, the period of a plasma concentration within the therapeutic range of the at least one second drug may be coextensive with at least about 80% of the period over which the dosage form provides a plasma concentration within the therapeutic range of the first drug.

In yet another aspect, the dosage form may comprise a bi-layered tablet.

In a still further aspect of the dosage form, the first plasma half-life may be at least about 8 hours.

In another aspect, the dosage form may be associated with instructions to administer the dosage form about three or fewer times per day, e.g., 1, 2 or 3 times per day.

The pharmaceutical dosage forms of the present invention which are set forth above and constitute one aspect of the present invention comprise promethazine and/or one or more pharmaceutically acceptable salts thereof. The preferred salt of promethazine is the hydrochloride. However, other pharmaceutically acceptable salts of promethazine may be used as well. The term “pharmaceutically acceptable salt” as used herein and in the appended claims refers to those salts of a particular drug that are not substantially toxic at the dosage administered to achieve the desired effect and do not independently possess significant pharmacological activity. The salts included within the scope of this term are pharmaceutically acceptable acid addition salts of a suitable inorganic or organic acid. Non-limiting examples of suitable inorganic acids are, for example hydrochloric, hydrobromic, sulfuric and phosphoric acids. Non-limiting examples of suitable organic acids include carboxylic acids, such as acetic, propionic, tannic, glycolic, lactic, pyruvic, malonic, succinic, fumaric, malic, tartaric, citric, cyclamic, ascorbic, nialeic, hydroxymaleic, benzoic, phenylacetic, 4-aminobenzoic, 4-hydroxybenzoic, anthranillic, cinnamic, salicylic, 4-aminosalicyclic, 2-phenoxybenzoic, 2-acetoxybenzoic and mandelic acids, as well as sulfonic acids, such as methanesulfonic, ethanesulfonic, and β-hydroxyethanesulfonic acids.

In addition to promethazine and/or pharmaceutically acceptable salt thereof the dosage forms of the present invention may contain one or more (e.g., one, two or three) second drugs. Preferred, non-limiting examples of such second drugs are decongestants (such as, e.g., phenylephrine, pseudoephedrine and pharmaceutically acceptable salts thereof), antitussives (such as, e.g., codeine, dihydrocodeine, hydrocodone, dextromethorphan and pharmaceutically acceptable salts thereof), expectorants and mucus thinning drugs (such as, e.g., guaifenesin), analgesics (such as, e.g., aspirin, acetaminophen, ibuprofen, ketoprofen, naproxen, sodium naproxen, meloxicam, hydrocodone, oxycodone, morphine, meperidine, and fentanyl) and antihistamines (such as, e.g., chlorpheniramine, carbinoxamine and pharmaceutically acceptable salts thereof). Particularly preferred as second drugs are pseudoephedrine and pharmaceutically acceptable salts thereof, in particular, pseudoephedrine hydrochloride.

The dosage forms of the present invention are capable of providing a promethazine plasma concentration within the therapeutic range for at least about 24 hours per single dose. Additionally or alternatively, the dosage forms of the present invention which contain at least one second drug are capable of providing a plasma concentration within the therapeutic range of the at least one second drug over a period which is coextensive with (overlaps) at least about 70%, more preferred at least about 80%, e.g., at least about 90%, at least about 95%, or about 100%, of the period over which the dosage form provides a plasma concentration within the therapeutic range of the promethazine. The term “therapeutic range” as used herein and in the appended claims refers to the range of drug levels (including active metabolite levels) within which most patients will experience a significant therapeutic effect (including alleviation of symptoms) without an undesirable degree of adverse reactions. It is noted that the term “coextensive with” does not exclude, but rather includes, cases where a part of the period over which the plasma concentration of the at least one second drug (and/or active metabolites thereof) is within the therapeutic range is outside the period over which the plasma concentration of the promethazine is within the therapeutic range. In other words, even if the corresponding period for the at least one second drug is to overlap, for example, 70% of the corresponding period of the promethazine, a certain percentage (preferably not more than about 30%, e.g., not more than about 20%, not more than about 10% or even not more than about 5%) of the total period over which the plasma concentration of the at least one second drug is within the therapeutic range may be outside the period over which the plasma concentration of the promethazine is within the therapeutic range, and vice versa.

The period over which the therapeutic range of a particular drug may be provided in a given case depends, at least in part, on the plasma half-life of the drug and/or active metabolites thereof. The term “plasma half-life” as used herein refers to the time required for the plasma drug concentration to decline by 50%. The shorter the plasma half-life of a particular drug, the shorter will be the period within the therapeutic range of the drug which is provided by a single administered dose of the drug. In one preferred aspect of the dosage form of the present invention, the plasma half-life of the at least one second drug will be shorter than the plasma half-life of the promethazine by at least about 3 hours, e.g., by at least about 4 hours, by at least about 5 hours, by at least about 6 hours, by at least about 8 hours, or even by at least about 10 hours.

A preferred, although non-limiting, embodiment of the dosage forms of the present invention is a tablet, in particular, a bi-layered tablet. Non-limiting examples of other embodiments of the dosage form of the invention are capsules, pills, chewable tablets, suspensions, solutions, gels, syrups, and suppositories.

The bi-layered tablet which forms one aspect of the present invention comprises two layers. The first layer comprises promethazine and/or a pharmaceutically acceptable salt thereof, as discussed above. The second layer comprises promethazine and/or a pharmaceutically acceptable salt thereof and/or (i.e., additionally or alternatively) at least one additional drug which preferably is selected from decongestants, antitussives, expectorants, mucus thinning drugs, analgesics and antihistamines. Specific and non-limiting examples of such drugs are given above. If at least one additional drug is present, the bi-layered tablet provides a plasma concentration within the therapeutic range of the at least one additional drug over a period which is coextensive with at least about 70%, preferably at least about 80%, e.g., at least about 90% or even about 100% of the period over which the bi-layered tablet provides a plasma concentration within the therapeutic range of the promethazine.

In a preferred aspect of the bi-layered tablet, the promethazine and/or pharmaceutically acceptable salt thereof is the only active ingredient in the first layer. The second layer will usually contain promethazine and/or a pharmaceutically acceptable salt thereof and/or one, two, three or even more additional drugs.

In another preferred aspect of the bi-layered tablet, the first layer is an immediate release layer and the second layer is a controlled release layer. The term “controlled release layer” as used herein and in the appended claims refers to any layer that is not an immediate release layer, i.e., does not release all of the active ingredients contained therein within a relatively short time (for example, within not more than about 1 hour, e.g., within not more than about 0.5 hours, following ingestion of the dosage form). Accordingly, this term is a generic term which encompasses, e.g., sustained (extended) release layers, pulsed release layers, delayed release layers, and the like. Preferably, the controlled release layer releases the one or more active ingredients contained therein continuously or intermittently and, preferably, in approximately equal amounts per time unit (e.g., zero order release rate), over an extended period of time such as, e.g., at least about 2 hours, at least about 3 hours, at least about 4 hours, at least about 6 hours, at least about 8 hours, at least about 10 hours, or at least about 12 hours. The desirable length of the time period of continuous or intermittent (e.g., pulsed) release depends, inter alia, on the plasma half-life of the drug and/or an active metabolite thereof. Also, especially if both an immediate release layer and a controlled release layer comprise promethazine and/or a pharmaceutically acceptable salt thereof, the controlled release layer may be a delayed release layer which starts releasing the drug only after a predetermined period of time (e.g., all at once in the fashion of an immediate release layer or in a controlled manner such as, e.g., at a constant rate, intermittently, etc.).

The first layer of the bi-layered tablet of the present invention will usually contain at least about 10 mg, e.g., at least about 15 mg, at least about 20 mg, or at least about 25 mg of promethazine hydrochloride or an equivalent amount (in terms of molar amount) of promethazine and/or any other pharmaceutically acceptable salt thereof. Usually, the first layer will not contain more than about 100 mg, e.g., not more than about 90 mg, not more than about 70 mg, or not more than about 50 mg of promethazine hydrochloride or an equivalent amount of promethazine and/or any other pharmaceutically acceptable salt thereof.

The second layer of the bi-layered tablet preferably is a controlled release layer, in particular, a sustained release layer. The controlled release layer may contain, by way of non-limiting example, (i) promethazine hydrochloride, usually in an amount which is not less than about 10 mg, e.g., not less than about 20 mg, or not less than about 30 mg, not less than about 40 mg, but not more than about 90 mg, e.g., not more than about 80 mg, not more than about 70 mg, or not more than about 50 mg, or equivalent amounts of any other pharmaceutically acceptable salt of promethazine; and/or (ii) phenylephrine hydrochloride, usually in an amount which is not less than about 1 mg, e.g., not less than about 10 mg, or not less than about 15 mg, but not more than about 90 mg, e.g., not more than about 50 mg, or not more than about 25 mg, or equivalent amounts of any other pharmaceutically acceptable salt of phenylephrine; and/or (iii) pseudoephedrine hydrochloride, usually in an amount which is not less than about 10 mg, e.g., not less than about 25 mg, not less than about 40 mg, or not less than about 80 mg, but not more than about 240 mg, e.g., not more than about 220 mg, not more than about 200 mg, or not more than about 180 mg, or equivalent amounts of any other pharmaceutically acceptable salt of pseudoephedrine. In a particularly preferred aspect, the second layer will comprise at least pseudoephedrine and/or a pharmaceutically acceptable salt thereof. Also, for a once-a-day dosage form, the amounts of the individual drugs will usually be at the upper end of the ranges recited above.

Another aspect of the present invention is a multi-layered tablet which comprises at least a first layer and a second layer, but may optionally comprise a third, fourth, fifth, etc. layer. The first layer, which preferably is an immediate release layer, comprises promethazine and/or a pharmaceutically acceptable salt thereof (preferably as the only active ingredient contained therein) and the mandatory second layer is a controlled release layer and may comprise promethazine and/or a pharmaceutically acceptable salt thereof and/or at least one drug which is selected from decongestants, antitussives, expectorants, mucus thinning drugs, analgesics and antihistamines. If more than one additional drug is to be incorporated in the tablet, the second layer may contain all of the additional drugs. Alternatively, a separate (third) layer may be provided for the second additional drug, for example, in cases where it would be difficult to design a controlled release layer which provides a desired release rate for both the first and the second additional drug. Of course, a fourth, fifth, etc. layer may be provided for a third or fourth additional drug, and so on. Alternatively and by way of non-limiting example, the second and a third layer may contain the same drug or drugs, but in different (relative) concentrations and/or incorporated in a different controlled release formulation.

The multi-layered tablet of the present invention will usually be made up of two or more distinct layers or discrete zones of granulation compressed together with the individual layers lying on top of one another. Layered tablets have the appearance of a sandwich because the edges of each layer or zone are exposed. Such conventional layered tablets are generally prepared by compressing a granulation onto a previously compressed granulation. The operation may be repeated to produce multi-layered tablets of more than two layers. In a preferred embodiment of the multi-layered tablet of the present invention, the tablet consists of two layers.

It is to be noted that it is not necessary for the two or more individual layers of the multi-layered tablet of the present invention to lie on top of one another. By way of non-limiting example, a second layer (e.g., sustained release layer) may be partially or completely surrounded by a first layer (e.g., an immediate release layer). For example, the second layer may be coated with the first layer. In the case of three layers, for example, the third layer may be partially or completely coated with the second layer, which in turn may be partially or completely coated with the first layer. Of course, these are but a few examples of the many different ways in which the various layers of the multi-layered tablet of the present invention can be arranged relative to each other. Moreover, it is to be understood that the tablets of the present invention are not limited to such multi-layered tablets. By way of non-limiting example, the tablet may comprise an immediate release matrix which comprises promethazine and/or a pharmaceutically acceptable salt thereof, which matrix has dispersed therein particles of one or more sustained release formulations which have promethazine and/or a pharmaceutically acceptable salt thereof and/or any of the other desired drug(s) incorporated therein.

There are many other ways of combining formulations which provide different release profiles, and all of these other ways are contemplated by the present invention. For example, granules of immediate release formulation and granules of controlled release formulation (e.g., granules of immediate release formulation which are provided with an extended release coating) may be incorporated in a single capsule in a desired ratio.

In another aspect of the multi-layered tablet, the at least one drug in the second layer (and/or in the additional layers) may have a plasma half-life which is shorter by at least about 3 hours, e.g., shorter by at least about 4 hours, or shorter by at least about 6 hours, than the plasma half-life of promethazine and/or pharmaceutically acceptable salt thereof.

In another aspect of the multi-layered tablet, the tablet may provide a plasma concentration within a therapeutic range of the at least one drug in the second layer (e.g., one or both of phenylephrine, pseudoephedrine and pharmaceutically acceptable salts thereof) over a period which is coextensive with at least about 80%, e.g., at least about 90%, of the period over which the multi-layered tablet provides a plasma concentration within the therapeutic range of the promethazine in the first layer. Preferably, this coextensive period is at least about 18 hours, e.g., at least about 20 hours, at least about 22 hours, or at least about 24 hours.

Another aspect of the present invention is formed by a liquid dosage form, preferably a suspension, which comprises (a) promethazine and/or a pharmaceutically acceptable salt thereof and (b) promethazine and/or a pharmaceutically acceptable salt thereof, and/or at least one drug which is selected from decongestants, expectorants, mucus thinning drugs, antitussives, analgesics and antihistamines. If the at least one other drug is present, this liquid dosage form provides a plasma concentration within the therapeutic range of the at least one other drug over a period which is coextensive with at least about 70%, preferably at least 80%, e.g., at least 90%, of the period over which the liquid dosage form provides a plasma concentration within the therapeutic range of component (a). By way of non-limiting example, component (b) may be incorporated into a solid controlled release formulation. For example, particles of component (b) may be provided with a controlled release coating (e.g. a controlled release coating comprising an organic polymer such as, e.g., a polyacrylate). This formulation may then be comminuted, if necessary, in an appropriate manner (e.g., by milling) to form particles of a size which is small enough to be suitable for being suspended in a pharmaceutically acceptable liquid carrier. Component (a), on the other hand, may be used as such or incorporated in a solid immediate release formulation, comminuted and incorporated into the liquid carrier as well. A non-limiting example of a corresponding procedure is described in the Examples below.

Prior to incorporating components (a) and (b) into a pharmaceutically acceptable liquid carrier to form a liquid dosage form according to the present invention at least a part of component (a) and/or at least a part of component (b) may be converted into a complex with a complexing agent. Non-limiting examples of suitable complexing agents comprise ion-exchange resins such as, e.g., (sodium) polystyrene sulfonate.

The dosage forms of the present invention can be manufactured by processes which are well known to those of skill in the art. For example, for the manufacture of bi-layered tablets, the active ingredients may be dispersed uniformly into a mixture of excipients, for example, by high shear granulation, low shear granulation, fluid bed granulation, or by blending for direct compression. Excipients may include diluents, binders, disintegrants, dispersants, lubricants, glidants, stabilizers, surfactants and colorants. Diluents, also termed “fillers”, are typically used to increase the bulk of a tablet so that a practical size is provided for compression. Non-limiting examples of diluents include lactose, cellulose, microcrystalline cellulose, mannitol, dry starch, hydrolyzed starches, powdered sugar, talc, sodium chloride, silicon dioxide, titanium oxide, dicalcium phosphate dihydrate, calcium sulfate, calcium carbonate, alumina and kaolin. Binders impart cohesive qualities to a tablet formulation and are used to ensure that a tablet remains intact after compression. Non-limiting examples of suitable binders include starch (including corn starch and pregelatinized starch), gelatin, sugars (e.g., glucose, dextrose, sucrose, lactose and sorbitol), celluloses, polyethylene glycol, waxes, natural and synthetic gums, e.g., acacia, tragacanth, sodium alginate, and synthetic polymers such as polymethacrylates and polyvinylpyrrolidone. Lubricants facilitate tablet manufacture; non-limiting examples thereof include magnesium stearate, calcium stearate, stearic acid, glyceryl behenate, and polyethylene glycol. Disintegrants facilitate tablet disintegration after administration, and non-limiting examples thereof include starches, alginic acid, crosslinked polymers such as, e.g., crosslinked polyvinylpyrrolidone, croscarmellose sodium, potassium or sodium starch glycolate, clays, celluloses, starches, gums and the like. Non-limiting examples of suitable glidants include silicon dioxide, talc and the like. Stabilizers inhibit or retard drug decomposition reactions, including oxidative reactions. Surfactants may be anionic, cationic, amphoteric or nonionic. If desired, the tablets may also contain minor amounts of nontoxic auxiliary substances such as pH buffering agents, preservatives, e.g., antioxidants, wetting or emulsifying agents, solubilizing agents, coating agents, flavoring agents, and the like.

Extended/sustained release formulations may be made by choosing the right combination of excipients that slow the release of the active ingredients by coating or temporarily bonding or decreasing the solubility of the active ingredients. Examples of these excipients include cellulose ethers such as hydroxypropylmethylcellulose (e.g., Methocel K4M), polyvinylacetate-based excipients such as, e.g., Kollidon SR, and polymers and copolymers based on methacrylates and methacrylic acid such as, e.g., Eudragit NE 30D.

There are several commercially available tablet presses capable of making bi-layered tablets. For example, Manesty RotaPress Diamond, a 45 station D tooling press, is capable of making bi-layered tablets described in this application. Non-limiting examples of presses for the manufacture of bi-layered tablets include Fette America Model No. PT 3090; Maneklal Global Exports (Mumbai, India) Models JD and DH series; Niro Pharma Systems, Model R292F; and Korsch AG Models XL 800 and XL 400.

### DETAILED DESCRIPTION OF THE PRESENT INVENTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description making apparent to those skilled in the art how the several forms of the present invention may be embodied in practice.

### EXAMPLE 1

Once-A-Day Dosage Form

A bi-layered tablet for administration every 24 hours which comprises 100 mg of promethazine hydrochloride is illustrated as follows:

| | | Dose | w/w |
|---|---|---|---|
| Ingredient | Process Step | (in mg) | (in %) |
| Calcium Phosphate Dibasic Anhyd. | Dry Mix | 25.00 | 7.1 |
| Prosolv SMCC 90 | Dry Mix | 25.00 | 7.1 |
| Promethazine HCl | Dry Mix- | 100.00 | 28.6 |
| | Active | | |
| Purified Water | Granulation | 35.00 | 0.0 |
| | Mix | | |
| Povidone K-30 | Granulation | 10.00 | 2.9 |
| | Mix | | |
| Methocel K4M Premium | Post Mix | 55.00 | 17.1 |
| Methocel K4M Premium | Final Blend | 110.00 | 35.7 |
| Stearic Acid NF | Final Blend | 5.00 | 1.4 |
| Microcrystalline Cellulose | Final Blend | 10.00 | 2.9 |
| Magnesium Stearate NF | Lube Blend | 5.00 | 1.4 |
| FD&C Blue #1 Alum Lake | Lube Blend | 5.00 | 1.4 |

| | | |
|---|---|---|
| TOTAL | 350.00 | 100.0 |

Manufacturing Process:

The active ingredients and excipients are mixed in a high shear mixer/granulator for about 10 minutes. The granulation solution, water and PovidoneK30 (polyvinyl pyrrolidone) are pumped into the contents of the granulator to wet the powder. After the solution is added the post mix sustained release matrix ingredient Methocel K4M premium (hydroxypropyl methylcellulose) is added and the contents are mixed until a uniform granulation is developed. Upon completion of the granulation process, the wet mass is dried at 45° C. The dried granulation powder is milled using a Fitzmill and blended in a V-Blender with other excipients. The dye is added to one half of the granulation powder. This granulation material in powder form is ready for compression on a bi-layer tablet press. Final tablet weight is 350 mg.

### EXAMPLE 2

Once-A-Day Dosage Form

A bi-layered tablet for once-a-day administration which comprises 100 mg of promethazine hydrochloride in an immediate release layer and 240 mg of pseudoephedrine hydrochloride in a sustained release layer is illustrated as follows:

### Immediate Release Layer

| | Dose | |
|---|---|---|
| INGREDIENTS | (mg) | % by Weight |
| PROMETHAZINE HCL | 100.000 | 33.33 |
| CALCIUM PHOSPHATE DIBASIC | 75.000 | 25.00 |
| USP DIHYDRATE | | |
| PROSOLV SMCC 90 | 41.000 | 13.67 |
| EUDRAGIT RS 30 D | 60.000 | 6.00 |
| METHOCEL K4M PREM USP | 18.750 | 6.25 |
| METHOCEL K4M PREM USP | 39.000 | 13.00 |
| COLLOIDAL SILICON DIOXIDE | 2.250 | 0.75 |
| STEARIC ACID | 3.000 | 1.00 |
| MAGNESIUM STEARATE | 3.000 | 1.00 |
| TOTAL | 300.000 | 100.00 |

### Sustained Release Layer

| | Dose | |
|---|---|---|
| INGREDIENTS | (mg) | % by Weight |
| PSEUDOEPHEDRINE HCL | 240.000 | 53.55 |
| CALCIUM PHOSPHATE DIBASIC | 73.000 | 16.222 |
| USP DIHYDRATE | | |
| FD&C BLUE #1 ALM LAKE | 0.645 | 0.15 |
| PROSOLV SMCC 90 | 46.000 | 10.22 |
| PURIFIED WATER | 54.000 | 0.00 |
| POVIDONE K-30 USP | 13.500 | 3.00 |
| PURIFIED WATER | 5.000 | 0.00 |
| METHOCEL K4M PREM USP | 22.500 | 5.00 |
| METHOCEL K4M PREM USP | 44.650 | 9.92 |
| FD&C BLUE #1 ALM LAKE | 0.675 | 0.15 |
| STEARIC ACID | 4.500 | 1.00 |
| MAGNESIUM STEARATE | 4.500 | 1.00 |
| TOTAL | 450.000 | 100.00 |

Manufacturing process:

Promethazine HCl (Immediate Release Layer)
1. Blend the dry mix amounts of Promethazine HCl, Calcium Phosphate Dibasic Dihydrate, and Prosolv SMCC 90 with a high sheer mixer/granulator for 10 minutes.
2. With the mixer/granulator on, pump the Eudragit RS 30D into the mixer/granulator. After completion, stop the mixer/granulator and rinse the container with Purified Water. Pump the rinse water to the mixer/granulator with mixer on. Turn off mixer when completed.
3. Charge the post mix amount of Methocel K4M premium to the mixer/granulator and mix for 1 minute.
4. Dry the granulation until the LOD (loss on Drying) is 4% or less.
5. Resize the dried granulation through a number 14 mesh screen.
6. Mix approximately 30% of the remaining Methocel K4M premium with the Colloidal Silicon Dioxide in an appropriate bag. Screen through a 30 mesh screen.
7. Screen the remaining amount of Methocel K4M premium through a number 14 mesh screen.
8. Blend the screened materials from steps 5, 6 and 7 using a V-blender for 20 minutes.
9. Screen the Magnesium Stearate using a number 30 mesh screen.
10. Transfer the screened Magnesium Stearate to the V-blender and blend for 3 minutes. When completed discharge and set aside for step 11 below.

Pseudoephedrine HCl (Sustained Release Layer)
1. Prepare a solution using the Povidone K-30, and Purified Water.
2. Blend the Pseudoephedrine HCl, Calcium Phosphate Dibasic USP Dihydrate, FD&C Blue #1 Aluminum Lake, and Prosolv SMCC 90 with a high sheer mixer/granulator for 10 minutes.
3. With the mixer/granulator on, pump the solution prepared in step 1 in to the mixer/granulator. After completion, stop the mixer/granulator and rinse the container with Purified Water. Pump the rinse water to the mixer/granulator with mixer on. Allow mixture to mix for an additional minute then turn mixer/granulator off.
4. Charge the post mix amount of Methocel K4M premium to the mixer/granulator and mix for 1 minute.
5. Dry the granulation until the LOD is 4% or less.
6. Resize the dried granulation through a number 14 mesh screen.
7. Screen the final blend amount of Methocel K4M premium through a number 14 mesh screen.
8. Screen the final blend amounts of Stearic Acid and FD&C Blue #1 Aluminum Lake through a number 30 mesh screen.
9. Blend the screened materials from step 6, 7, and 8 using a V-blender for 20 minutes.
10. Screen the lube amount of Magnesium Stearate using a number 30 mesh screen.
11. Transfer the screened Magnesium Stearate to the V-blender and blend for 3 minutes. When completed discharge and set aside for step 10.
12. Manufacture bi-layered tablets using a rotary bi-layer tablet press where in each tablet layer 1 is 300.0 mg and layer 2 is 450.0 mg.

### EXAMPLE 3

Bi-Layered Tablet (Direct Compression)

A bi-layered tablet in accordance with the present invention which comprises promethazine hydrochloride in one layer and phenylephrine hydrochloride and chlorpheniramine maleate in the other layer is illustrated as follows:

| | | |
|---|---|---|
| | Weight/tablet | Weight/1 kg batch |
| Ingredients | (mg) | (in grams) |

Layer 1 (Immediate release)

| | | |
|---|---|---|
| Promethazine Hydrochloride | 25.0 | 45.5 |
| Silicified Microcrystalline | 114.0 | 207.3 |
| Cellulose | | |
| Sodium Starch Glycolate | 10.0 | 18.2 |
| Magnesium Stearate | 1.0 | 1.8 |

Layer 2 (Sustained release)

| | | |
|---|---|---|
| Phenylephrine HCl | 20.0 | 36.4 |
| Chlorpheniramine Maleate | 8.0 | 14.5 |
| Lactose Monohydrate | 50.0 | 90.9 |
| Dicalcium Phosphate | 50.0 | 90.9 |
| Kollidon SR | 252.0 | 458.2 |
| Stearic acid | 15.0 | 27.3 |
| Magnesium Stearate | 5.0 | 9.1 |
| Total | 550.0 | 1000.0 |

Manufacturing Process

(a) Immediate release layer: Screen all ingredients through a USP sieve size # 30. Blend promethazine hydrochloride (45.5 gms), silicified microcrystalline cellulose (207.3 gms) and sodium starch glycolate (18.2 gms) in a twin shell blender for 20 minutes. Add magnesium stearate (1.8 gms), which acts as a lubricant, to the above blend and mix for 3 minutes.

(b) Sustained release layer: Screen all ingredients through a USP sieve size # 30. Preblend a portion of the Kollidon SR (145 gms) and all the chlorpheniramine maleate (14.5 gms) for 15 minutes. Add the remaining Kollidon SR (313.2 gms), phenylephrine hydrochloride (36.4 gms), lactose monohydrate (90.9 gms) and dicalcium phosphate (90.9 gms) to the above preblend and mix for an additional 20 minutes. Add stearic acid (27.3 gms) and magnesium stearate (9.1 gms) to the above blend and mix for three minutes.

Manufacture bi-layered tablets using a rotary bi-layer tablet press where in each tablet the immediate release layer is 150 mgs and the sustained release layer is 400 mgs.

By using the process described above, a bi-layered tablet of the following composition may be manufactured by using direct compression:

| | |
|---|---|
| | Weight/tablet |
| Ingredients | (mgs) |

Layer 1 (Immediate Release)

| | |
|---|---|
| Promethazine Hydrochloride | 50 |
| Silicified Microcrystalline Cellulose | 133.5 |
| Sodium Starch Glycholate | 15 |
| Magnesium Stearate | 1.5 |

Layer 2 (Sustained Release)

| | |
|---|---|
| Phenylephrine HCl | 20 |
| Chlorpheniramine Maleate | 8 |
| Lactose Monohydrate | 50 |
| Dicalcium Phosphate | 50 |
| Kollidon SR | 252 |
| Stearic Acid | 15 |
| Magnesium Stearate | 5 |
| Total | 600 |

### EXAMPLE 4

Bi-Layered Tablet (Wet Granulation):

A bi-layered tablet in accordance with the present invention which comprises promethazine hydrochloride in one layer and pseudoephedrine hydrochloride and chlorpheniramine maleate in the other layer is illustrated as follows:

| | | |
|---|---|---|
| | Weight/tablet | Weight/1 kg |
| Ingredients | (mgs) | batch (gms) |

Layer 1 (Immediate release)

| | | |
|---|---|---|
| Promethazine Hydrochloride | 25.0 | 35.7 |
| Silicified Microcrystalline Cellulose | 111.0 | 158.6 |
| Povidone | 3.0 | 4.3 |
| Croscarmellose Sodium | 10.0 | 14.3 |
| Magnesium Stearate | 1.0 | 1.4 |

Layer 2 (Sustained release)

| | | |
|---|---|---|
| Pseudoephedrine HCl | 60.0 | 85.7 |
| Chlorpheniramine Maleate | 8.0 | 11.4 |
| Microcrystalline Cellulose (PH 102) | 30.0 | 42.9 |
| Lactose Monohydrate | 100.0 | 142.9 |
| Dicalcium Phosphate | 100.0 | 142.9 |
| Povidone | 15.0 | 21.4 |
| Methocel K4M Premium | 212.0 | 302.9 |
| Stearic Acid | 20.0 | 28.6 |
| Magnesium Stearate | 5.0 | 7.1 |
| Total | 700.0 | 1000.0 |

Manufacturing Process

(a) Immediate release layer: Screen all ingredients through a USP sieve size # 30. Blend promethazine hydrochloride (35.7 gms), silicified microcrystalline cellulose (158.6 gms) and croscarmellose sodium (14.3 gms) in a high shear mixer/granulator for 10 minutes. Granulate the above blend using a 30% povidone solution (4.3 gms povidone in 14.3 gms purified water). Dry the granulation until the loss on drying (LOD) is less than 2.0%. Screen the dried granulation through a USP sieve size # 14. Add the screened granulation and the prescreened magnesium stearate (1.4 gms) to the above blend and mix for 3 minutes.

(b) Sustained release layer: Screen all ingredients through a USP sieve size # 30. Blend the pseudoephedrine hydrochloride (87.5 gms), chlorpheniramine maleate (11.4 gms), microcrystalline cellulose PH 102 (42.9 gms), lactose monohydrate (142.9 gms), dicalcium phosphate (142.9 gms), Methocel K4M Premium (302.9 gms) and stearic acid (28.6 gms) in a high shear mixer/granulator for 10 minutes. Granulate the above blend using a 30% povidone solution (21.4 gms povidone in 71.3 gms purified water). Dry the granulation till the LOD is less than 2.0%. Screen granules through a USP sieve size # 14. Add granules and the prescreened magnesium stearate (7.1 gms) to the above blend and mix for 3 minutes.

Manufacture bi-layered tablets using a rotary bi-layer tablet press where in each tablet the immediate release layer is 150 mgs and the sustained release layer is 550 mgs.

By using the process described above, a bi-layered tablet of the following composition may be manufactured by using wet granulation:

| | |
|---|---|
| | Weight/tablet |
| Ingredients | (mgs) |

Layer 1 (Immediate Release)

| | |
|---|---|
| Promethazine Hydrochloride | 50 |
| Silicified Microcrystalline cellulose | 129.5 |
| Povidone | 4 |
| Croscarmellose sodium | 15 |
| Magnesium Stearate | 1.5 |

Layer 2 (Sustained Release)

| | |
|---|---|
| Pseudoephedrine HCl | 60 |
| Chlorpheniramine Maleate | 8 |
| Microcrystalline Cellulose 102 | 30 |
| Lactose Monohydrate | 100 |
| Dicalcium Phosphate | 100 |
| Povidone | 15 |
| Hydroxypropylmethylcellulose | 212 |
| Stearic Acid | 20 |
| Magnesium Stearate | 5 |
| Total | 750 |

The above examples illustrate how to manufacture a bi-layered tablet containing promethazine hydrochloride in one layer and a combination of an antihistamine and/or a decongestant and/or an antitussive and/or an expectorant. For the layer that does not contain promethazine hydrochloride, combinations of one or more each of the non-limiting examples of possible ingredients in an exemplary range as described in the following Table 1 can be made depending on the specific therapeutic effect desired.

TABLE 1

| | | | Preferred | | OTC | |
|---|---|---|---|---|---|---|
| | Amount per | | Amount per | | Daily | |
| Active ingredient | Tablet | | Tablet | | Dosage | |
| ANTIHISTAMINES | | | | | | |
| Azelastine hydrochloride | 0.1-2.0 | mg | 0.125 | mg | | |
| Azatadine hydrochloride | 0.1-4.0 | mg | 1 | mg | | |
| Brompheniramine maleate | 0.1-64 | mg | 2-16 | mg | 24 | mg |
| Dexbrompheniramine | 0.1-24 | mg | 3-6 | mg | 12 | mg |
| maleate | | | | | | |
| Carbinoxamine maleate | 0.1-16 | mg | 4 | mg | | |
| Cetirizine hydrochloride | 0.1-40 | mg | 5-10 | mg | | |
| Chlorcyclizine | 0.1-300 | mg | | | 75 | mg |
| Chlorpheniramine maleate | 0.1-64 | mg | 2-16 | mg | 24 | mg |
| Chlorpheniramine polistirex | 0.1-32 | mg | 4-8 | mg | | |
| Clemastine | 0.1-12 | mg | 0.5-2.68 | mg | | |
| Cyproheptadine | 0.1-16 | mg | 2-4 | mg | | |
| Dexchlorpheniramine | 0.1-24 | mg | 2 | mg | 12 | mg |
| maleate | | | | | | |
| Cyproheptadine | 0.1-32 | mg | 2-4 | mg | | |
| hydrochloride | | | | | | |
| Diphenhydramine | 0.1-300 | mg | 10-50 | mg | 300 | mg |
| hydrochloride | | | | | | |
| Diphenhydramine citrate | 0.1-2000 | mg | | | 456 | mg |
| Bromodiphenhydramine | 0.1-200 | mg | 12.5-25 | mg | | |
| hydrochloride | | | | | | |
| Doxylamine succinate | 0.1-200 | mg | 12.5-25 | mg | 75 | mg |
| Fexofenadine hydrochloride | 0.1-720 | mg | 30-180 | mg | | |
| Hydroxyzine hydrochloride | 0.1-400 | mg | 10-100 | mg | | |
| Hydroxyzine pamoate | 0.1-400 | mg | 25-100 | mg | | |
| Loratadine | 0.1-80 | mg | 1-10 | mg | | |
| Desloratadine | 0.1-40 | mg | 5 | mg | | |
| Phenindamine tartrate | 0.1-750 | mg | | | 150 | mg |
| Pheniramine maleate | 0.1-750 | mg | | | 150 | mg |
| Pyrilamine maleate | 0.1-200 | mg | 25 | mg | 200 | mg |
| Terfenadine | | | | | | |
| Thenyldiamine | | | | | | |
| Thonzylamine | 0.1-3000 | mg | | | 600 | mg |
| Thymol | | | | | | |
| Tripelennamine | 0.1-400 | mg | 25-50 | mg | | |
| hydrochloride | | | | | | |
| Triprolidine hydrochloride | 0.1-40 | mg | 1.25-5 | mg | 10 | mg |
| ANTITUSSIVES | | | | | | |
| Chlorphedianol | 0.1-800 | mg | | | 100 | mg |
| hydrochloride | | | | | | |
| Codeine | 0.1-240 | mg | 8.4-60 | mg | 120 | mg |
| Codeine phosphate | 0.1-240 | mg | 2.5-60 | mg | 120 | mg |
| Codeine sulfate | 0.1-480 | mg | | | 120 | mg |
| Dextromethorphan | 0.1-480 | mg | | | 120 | mg |
| Dextromethorphan | 0.1-240 | mg | 3.3-30 | mg | 120 | mg |
| hydrobromide | | | | | | |
| Dextromethorphan | 0.1-240 | mg | 30 | mg | | |
| polistirex | | | | | | |
| Diphenhydramine citrate | 0.1-1000 | mg | | | 228 | mg |
| Diphenhydramine | 0.1-400 | mg | 10-50 | mg | 150 | mg |
| hydrochloride | | | | | | |
| Benzonatate | 0.1-800 | mg | 100-200 | mg | | |
| Hydrocodone bitatrate | 0.1-40 | mg | 1.66-10 | mg | | |
| Dihydrocodeine | 0.1-128 | mg | 16-32 | mg | | |
| Caramiphen edisylate | 0.1-160 | mg | 6.7-40 | mg | | |
| Carbetapentane tannate | 0.1-480 | mg | 30-60 | mg | | |
| Carbetapentane citrate | 0.1-160 | mg | 20 | mg | | |
| Hydromorphone | 0.1-8 | mg | 1 | mg | | |
| Noscapine | 0.1-200 | mg | | | | |
| EXPECTORANT | | | | | | |
| Guaifenesin | 0.1-2000 | mg | 50-1200 | | 2400 | mg |

### REFERENCE EXAMPLE

Extended Release Suspension

| Ingredients | Amount/5 ml |
|---|---|
| Hydrocodone ion-exchange complex | Equivalent to 8 mgs |
| | Hydrocodone bitartarate |
| Dexchlorpheniramine ion-exchange complex | Equivalent to 4 mgs |
| | Dexchlorpheniramine |
| | maleate |
| Phenylephrine ion-exchange complex | Equivalent to 10 mgs |
| | Phenylephrine HCl |
| Eudragit ® L 100 | 0.2 to 2.8 grams |
| Glycerin | 315 mgs |
| Polysorbate 80 | 1.5 mgs |
| Carbomer (e.g., Carbopol ® 974) | 15 mgs |
| Methyl Paraben | 9 mgs |
| Propyl Paraben | 1 mgs |
| Artificial grape flavor | 5 mgs |
| FD&C red # 40 dye | 0.5 mgs |
| Water | q.s |

The formula described above serves as a non-limiting example. Any active drug which is in the form of a salt, such as promethazine hydrochloride, codeine phosphate, pseudoephedrine hydrochloride, morphine sulfate, or meperidine hydrochloride can be incorporated as an ion-exchange resin complex.

Procedure

(1) Add the appropriate amount of sodium polystyrene sulphonate USP (e.g. Amberlite® IRP 69) to a hydrocodone bitartarate, dexchlorpheniramine maleate and phenylephrine HCl solution.
(2) Stir the mix for 12 hrs to allow complete drug/resin complex formation.
(3) Separate and dry the insoluble drug/resin complex.
(4) Granulate the drug/resin complex with a delayed release/enteric polymer (e.g. Eugragit® L 100, Kollidon® MAE, Aquacoa®t cPD) and dry the granules.
(5) Mill the granules, if needed.
(6) To an appropriate amount of water add the following ingredients and dissolve: Carbomer (e.g., Carbopol® 974), glycerin, polysorbate 80, methyl paraben, propyl paraben, artificial grape flavor, FD&C red # 40 dye.
(7) Add milled granules.
(8) Add water to make up to a final volume.
(9) Agitate at suitable rate to avoid settling of the suspension and maintain a homogeneous product mixture.
(10) Fill in suitable containers ensuring that the product is homogeneous throughout the filling operation.

It is noted that the foregoing examples have been provided merely for the purpose of explanation and are in no way to be construed as limiting of the present invention. While the present invention has been described with reference to exemplary embodiments, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

1. A pharmaceutical dosage form which comprises at least one of promethazine and a pharmaceutically acceptable salt thereof, wherein the dosage form is capable of providing a promethazine plasma concentration within a therapeutic range for at least about 24 hours per single dose.

2. The dosage form of claim 1, wherein the dosage form is capable of providing relief from allergy symptoms in a patient in need thereof for at least about 24 hours per single dose.

3. The dosage form of claim 1, wherein the dosage form comprises at least two promethazine formulations which exhibit different release profiles.

4. The dosage form of claim 1, wherein the dosage form comprises an immediate release formulation.

5. The dosage form of claim 4, wherein the dosage form comprises a controlled release formulation.

6. The dosage form of claim 1, wherein the dosage form comprises a solid dosage form.

7. The dosage form of claim 6, wherein the solid dosage form is selected from tablets, capsules and caplets.

8. The dosage form of claim 1, wherein the dosage form comprises a bi-layered tablet.

9. The dosage form of claim 8, wherein the bi-layered tablet comprises an immediate release layer and a controlled release layer.

10. The dosage form of claim 9, wherein each of the immediate release layer and the controlled release layer comprises at least about 25 mg of promethazine hydrochloride or an equivalent amount of at least one other pharmaceutically acceptable salt of promethazine.

11. The dosage form of claim 8, wherein the bi-layered tablet comprises a total of at least about 70 mg of promethazine hydrochloride or an equivalent amount of at least one other pharmaceutically acceptable salt of promethazine.

12. The dosage form of claim 9, wherein at least one of the controlled release layer and the immediate release layer comprises at least about 40 mg of promethazine hydrochloride or an equivalent amount of at least one other pharmaceutically acceptable salt of promethazine.

13. The dosage form of claim 1, wherein the dosage form comprises at least about 70 mg of promethazine hydrochloride or an equivalent amount of at least one other pharmaceutically acceptable salt of promethazine.

14. The dosage form of claim 1, wherein the dosage form comprises at least about 90 mg of promethazine hydrochloride or an equivalent amount of at least one other pharmaceutically acceptable salt of promethazine.

15. The dosage form of claim 1, wherein the dosage form is capable of providing a promethazine plasma concentration within a therapeutic range within not more than about 1 hour following ingestion thereof.

16. The dosage form of claim 1, wherein the dosage form comprises at least one further drug.

17. The dosage form of claim 16, wherein the at least one further drug is selected from decongestants, antitussives, expectorants, mucus thinning drugs and analgesics.

18. The dosage form of claim 17, wherein the dosage form is capable of providing a plasma concentration of the at least one further drug within a therapeutic range for at least about 24 hours per single dose.

19. The dosage form of claim 18, wherein the at least one further drug is selected from one or more of phenylephrine, pseudoephedrine, phenylephrine, pseudoephedrine, codeine, dihydrocodeine, hydrocodone, dextromethorphan, carbetapentane, guaifenesin, acetaminophen, aspirin, ibuprofen, naproxen, oxycodone, morphine and hydromorphone and pharmaceutically acceptable salts thereof.

20. The dosage form of claim 16, wherein the dosage form is capable of providing a plasma concentration within a therapeutic range of promethazine over a period which is coextensive with at least about 70% of a period over which the dosage form is capable of providing a plasma concentration within a therapeutic range of the at least one further drug.

21. The dosage form of claim 20, wherein a plasma half-life of the at least one further drug is shorter than a plasma half-life of promethazine by at least about 3 hours.

22. The dosage form of claim 21, wherein a period of a plasma concentration within the therapeutic range of the at least one further drug is coextensive with at least about 90% of a period of a plasma concentration within the therapeutic range of promethazine

23. A pharmaceutical dosage form which comprises (a) at least one of promethazine and a pharmaceutically acceptable salt thereof in a first form or layer and (b) at least one of promethazine and a pharmaceutically acceptable salt thereof in a second form or layer which is different from the first form or layer, wherein the dosage form releases the promethazine (b) at least one of over a different period and at a different rate than the promethazine (a) and wherein the dosage form is capable of providing a promethazine plasma concentration within a therapeutic range for at least about 24 hours per single dose.

24. The dosage form of claim 23, wherein the dosage form is a multi-layered tablet which comprises at least one immediate release layer and at least one controlled release layer and at least one of these layers comprises at least one of promethazine and a pharmaceutically acceptable salt thereof, and wherein at least one of the layers of the multi-layered tablet comprises at least one further drug.

25. A pharmaceutical dosage form which comprises at least one of promethazine and a pharmaceutically acceptable salt thereof and at least one of pseudoephedrine and a pharmaceutically acceptable salt thereof, wherein the dosage form is capable of providing plasma concentrations within therapeutic ranges of both promethazine and pseudoephedrine for at least about 24 hours per single dose.

26. The dosage form of claim 25, wherein the dosage form comprises a solid dosage form.

27. The dosage form of claim 26, wherein the dosage form comprises a multi-layered tablet.

28. The dosage form of claim 25, wherein the dosage form is capable of providing promethazine and pseudoephedrine plasma concentrations within therapeutic ranges within not more than about 1 hour following ingestion thereof.

29. The dosage form of claim 28, wherein the dosage form comprises promethazine hydrochloride and pseudoephedrine hydrochloride.

30. The dosage form of claim 25, wherein the dosage form comprises at least about 70 mg of promethazine hydrochloride or an equivalent amount of at least one other pharmaceutically acceptable salt of promethazine.

31. The dosage form of claim 30, wherein the dosage form comprises at least about 180 mg of pseudoephedrine hydrochloride or an equivalent amount of at least one other pharmaceutically acceptable salt of pseudoephedrine per single dose.

32. The dosage form of claim 25, wherein the dosage form comprises at least one further drug.

33. The dosage form of claim 1, wherein the dosage form is associated with instructions to administer the dosage form once every 24 hours.

34. The dosage form of claim 23, wherein the dosage form is associated with instructions to administer the dosage form once every 24 hours.

35. The dosage form of claim 25, wherein the dosage form is associated with instructions to administer the dosage form once every 24 hours.

36. A method of alleviating a condition which can be alleviated by administration of promethazine, wherein the method comprises administering the pharmaceutical dosage form of claim 1 to a subject in need thereof.

37. The method of claim 36, wherein the condition that can be alleviated by administration of promethazine comprises an allergic reaction.

38. The method of claim 37, wherein the dosage form is administered not more than about once every 24 hours.

39. A method of alleviating one or more conditions which can be alleviated by administration of promethazine and by administration of a drug which is at least one of a decongestant, an antitussive, an expectorant, a mucus thinning drug and an analgesic, wherein the method comprises administering the dosage form of claim 17 to a subject in need thereof.

40. The method of claim 39, wherein the dosage form is administered not more than about once every 24 hours.

41. A method of alleviating one or more conditions which can be alleviated by administration of promethazine and pseudoephedrine, wherein the method comprises administering the dosage form of claim 25 to a subject in need thereof.

42. The method of claim 41, wherein the dosage form is administered not more than about once every 24 hours.

43. A process for making the pharmaceutical dosage form of claim 25, wherein the process comprises preparing a first composition which comprises promethazine or a pharmaceutically acceptable salt thereof and a second composition which comprises pseudoephedrine or a pharmaceutically acceptable salt thereof, and combining the first and second compositions.

44. The process of claim 43, wherein the compositions are combined by a method which comprises use of a tablet press.
